(19) United States
(12) Patent Application Publication
(10) Pub. No.: US 2024/0175594 A1
(43) Pub. Date: May 30, 2024

(54) SYSTEM FOR DETECTING AND CLEANING INDOOR AIR POLLUTION

(71) Applicant: MICROJET TECHNOLOGY CO., LTD., Hsinchu City (TW)

(72) Inventors: Hao-Jan MOU, Hsinchu City (TW); Chin-Chuan WU, Hsinchu City (TW); Yung-Lung HAN, Hsinchu City (TW); Chi-Feng HUANG, Hsinchu City (TW)

(21) Appl. No.: 18/161,986

(22) Filed: Jan. 31, 2023

(30) Foreign Application Priority Data
Nov. 25, 2022 (TW) 111145297

(51) Int. Cl. F24F 11/63; A61L 9/014; A61L 9/20; B01D 46/00; B01D 46/42; B01D 46/44; B01D 46/46; B01D 53/00; B01D 53/86; B01D 53/88; F24F 3/16; F24F 11/58; F24F 11/79

(52) U.S. Cl. CPC F24F 11/63; A61L 9/014; A61L 9/205; B01D 46/0041; B01D 46/429; B01D 46/442; B01D 46/46; B01D 53/007; B01D 53/8696; B01D 53/885; F24F 3/16; F24F 11/58; F24F 11/79; F24F 2110/65

(57) ABSTRACT

A system for detecting and cleaning indoor air pollution includes one or more outdoor gas detection devices, a plurality of indoor gas detection devices, a control central processor, and a plurality of filtering devices including one or more directing filtering devices. A directional blower is disposed on the directing filtering device and can be moved upwardly and downwardly, as well as rotating with respect to the directing filtering device. The indoor gas detection device is disposed on the directing filtering device, receiving the control command to enable the directing filtering device and to control the directional blower to be directed toward the air pollution location so as to generate a directed air convection. Therefore, the circulative filtration and the rapid clean of the air pollution can be achieved by the filtering component, allowing the indoor air pollution data to be a safety detection value.

### CROSS-REFERENCE TO RELATED APPLICATION

This non-provisional application claims priority under 35 U.S.C. § 119(a) to Patent Application No. 111145297 filed in Taiwan, R.O.C. on Nov. 25, 2022, the entire contents of which are hereby incorporated by reference.

### BACKGROUND

### Technical Field

The present invention relates to a system for detecting and cleaning indoor air pollution, in particular, to a system adapted to be utilized in an indoor space with a refresh air ventilation device or with a heating, ventilation and air conditioning system (HVAC system), thereby the circulative filtration and the rapid clean of the air pollution through a directed air convection can be achieved to allow the indoor air pollution data to approach to a non-detection state (almost zero), making the gas (air) of the indoor space to a safe and breathable state.

### Related Art

In light of people paying more and more attention to the ambient air quality in daily life, it is noted that the particulate matters (PM1, PM2.5, PM10), carbon dioxide, total volatile organic compounds (TVOC), formaldehyde and even particulates, aerogels, bacteria, viruses contained in the air might affect the human health, even might be life-threatening when exposure to these gases.

However, currently, it is not easy to control the indoor air quality since the affecting factors of the indoor air quality include not only the outdoor space air quality but also the air conditioning and the pollution sources in the indoor space (especially the dusts originated from poor circulation of air in the indoor space). Therefore, the air conditioners or air cleaners are utilized for improving the indoor air quality.

Consequently, for intelligently and quickly detect the indoor air pollution source, thereby effectively removing the air pollution from the indoor space, making the air into a safe and breathable state while the air quality in the indoor space is lowering a default standard, and monitoring the air quality of the indoor space whenever and wherever possible, it is an issue of the present invention to generate an air convection intelligently, as well as detecting and locating the air pollution rapidly, furthermore, to effectively control a directing filtering device to generate a directed air convection, thus the air pollution can be repeatedly filtered and rapidly cleaned through the directed air convection. Accordingly, the air pollution in the indoor space is cleaned to a safe and breathable state, allowing the air pollution data to approach to a non-detection state.

### SUMMARY

One object of the present invention is to provide a system for detecting and cleaning indoor air pollution, the system can be not only utilized in an indoor space with a refresh air ventilation device but also in an indoor space with an HVAC system. At least one outdoor gas detection device and a plurality of gas detection devices are utilized to detect and compare the indoor gas and the outdoor gas so as to determine whether the air pollution in the indoor space is to be exchanged and discharged to the outdoor space. Moreover, through the detection of the indoor gas detection devices in the filtering devices (for example, the ventilator, the cooker hood, the refresh air ventilation device, or the HVAC system) in the indoor space and the wireless transmission of the control central processor, the intelligent computation is performed to locate the air pollution location in the indoor space, and the control command is transmitted intelligently and selectively to enable the filtering devices (for example, the ventilator, the cooker hood, the refresh air ventilation device, or the HVAC system) to be driven to generate a certain directed air convection, thus the circulative filtration and the rapid clean of the air pollution can be achieved by the filtering component, allowing the indoor air pollution data to be a safety detection value in which the air pollution data approaches to a non-detection state (almost zero), and the gas (air) in the indoor space is cleaned to a safe and breathable state. Therefore, the air pollution in the indoor space can be filtered and cleaned instantly. Hence, a performance of locating the air pollution, guiding the air pollution, and cleaning and filtering the air pollution can be achieved.

In order to accomplish the above object(s), in the general embodiment of the present invention, a system for detecting and cleaning indoor air pollution includes at least one outdoor gas detection device, a plurality of indoor gas detection devices, a control central processor, and a plurality of filtering devices. The at least one outdoor gas detection device is configured to detect a qualitative property and a concentration of an air pollution of an outdoor gas and output an outdoor air pollution data. The indoor gas detection devices are disposed in an indoor space and configured to detect a qualitative property and a concentration of an air pollution in the indoor space and output an indoor air pollution data. The control central processor is configured to receive the outdoor air pollution data detected by the at least one outdoor gas detection device and the indoor air pollution data detected by the indoor gas detection devices, perform an intelligent computation to locate an air pollution location in the indoor space, and transmit a control command intelligently and selectively. Each of the filtering devices comprises at least one blower and at least one filtering component, furthermore, each of the filtering devices is provided with a corresponding one of the indoor gas detection devices. The indoor gas detection device disposed on each of the filtering device receives the control command to control the at least one blower to be driven to generate an air convection, and the air pollution in the indoor space is filtered by the at least one filtering component. The filtering devices comprise at least one directing filtering device. A directional blower is on the at least one directing filtering device and the directional blower is capable of being moved upwardly and downwardly, as well as rotating with respect to the at least one directing filtering device. The indoor gas detection device disposed on the at least one directing filtering device receives the control command to enable the operation of the at least one directing filtering device and control the directional blower to be directed toward the air pollution location in order to generate a directed air convection, therefore the circulative filtration and the rapid clean of the air pollution can be achieved by the at least one filtering component of the at least one directing filtering device, allowing the indoor air pollution data to be a safety detection value in which the air pollution data approaches to a non-detection state, and the air in the indoor space is cleaned to a safe and breathable state.

### BRIEF DESCRIPTION OF THE DRAWINGS

The invention will become more fully understood from the detailed description given herein below, the illustration is only for describing and thus not limitative of the invention, wherein:

FIG. 1A illustrates a schematic view (1) showing the operation of a system for detecting and cleaning indoor air pollution of an exemplary embodiment in the present invention, wherein the system is utilized in an indoor space;

FIG. 1B illustrates a schematic view (2) showing the operation of the system for detecting and cleaning indoor air pollution of the exemplary embodiment in the present invention, wherein the system is utilized in the indoor space;

FIG. 1C illustrates a schematic view of a refresh air ventilation device of the system for detecting and cleaning indoor air pollution shown in FIG. 1A;

FIG. 1D illustrates a schematic view of a heating, ventilation and air conditioning system (HVAC system) of the system for detecting and cleaning indoor air pollution shown in FIG. 1B;

FIG. 2A illustrates a schematic view of a blower and a filtering component of the filtering device of the system for detecting and cleaning indoor air pollution of the exemplary embodiment in the present invention;

FIG. 2B illustrates a schematic view of the filtering component of the system for detecting and cleaning indoor air pollution of the exemplary embodiment in the present invention;

FIG. 3A illustrates a perspective view of a directing filtering device of the system for detecting and cleaning indoor air pollution of the exemplary embodiment in the present invention;

FIG. 3B illustrates a schematic cross-sectional view showing the relative positional relationship among the components of the directing filtering device of the system for detecting and cleaning indoor air pollution of the exemplary embodiment in the present invention;

FIG. 4A illustrates a perspective view of a gas detection device of the system for detecting and cleaning indoor air pollution of the exemplary embodiment in the present invention;

FIG. 4B illustrates a perspective view (1) of a gas detection main body of the system for detecting and cleaning indoor air pollution of the exemplary embodiment in the present invention;

FIG. 4C illustrates a perspective view (2) of the gas detection main body of the system for detecting and cleaning indoor air pollution of the exemplary embodiment in the present invention;

FIG. 4D illustrates an exploded view of the gas detection device of the system for detecting and cleaning indoor air pollution of the exemplary embodiment in the present invention;

FIG. 5A illustrates a perspective view (1) of a base of the gas detection device of the system for detecting and cleaning indoor air pollution of the exemplary embodiment in the present invention;

FIG. 5B illustrates a perspective view (2) of the base of the gas detection device of the system for detecting and cleaning indoor air pollution of the exemplary embodiment in the present invention;

FIG. 6 illustrates a perspective view (3) of the base of the gas detection device of the system for detecting and cleaning indoor air pollution of the exemplary embodiment in the present invention;

FIG. 7A illustrates an exploded view of a piezoelectric actuator separating from the base of the gas detection device of the system for detecting and cleaning indoor air pollution of the exemplary embodiment in the present invention;

FIG. 7B illustrates a perspective view of the base in combination with the piezoelectric actuator of the gas detection device of the system for detecting and cleaning indoor air pollution of the exemplary embodiment in the present invention;

FIG. 8A illustrates an exploded view (1) of the piezoelectric actuator of the gas detection device of the system for detecting and cleaning indoor air pollution of the exemplary embodiment in the present invention;

FIG. 8B illustrates an exploded view (2) of the piezoelectric actuator of the gas detection device of the system for detecting and cleaning indoor air pollution of the exemplary embodiment in the present invention;

FIG. 9A illustrates a cross-sectional view (1) showing the operation of the piezoelectric actuator of the gas detection device of the system for detecting and cleaning indoor air pollution of the exemplary embodiment in the present invention;

FIG. 9B illustrates a cross-sectional view (2) showing the operation of the piezoelectric actuator of the gas detection device of the system for detecting and cleaning indoor air pollution of the exemplary embodiment in the present invention;

FIG. 9C illustrates a cross-sectional view showing the operation (3) of the piezoelectric actuator of the gas detection device of the system for detecting and cleaning indoor air pollution of the exemplary embodiment in the present invention;

FIG. 10A illustrates a cross-sectional view (1) of the gas detection main body of the gas detection device of the system for detecting and cleaning indoor air pollution of the exemplary embodiment in the present invention;

FIG. 10B illustrates a cross-sectional view (2) of the gas detection main body of the gas detection device of the system for detecting and cleaning indoor air pollution of the exemplary embodiment in the present invention;

FIG. 10C illustrates a cross-sectional view (3) of the gas detection main body of the gas detection device of the system for detecting and cleaning indoor air pollution of the exemplary embodiment in the present invention; and

FIG. 11 illustrates a schematic view showing the transmission of the gas detection device of the system for detecting and cleaning indoor air pollution of the exemplary embodiment in the present invention.

### DETAILED DESCRIPTION

The present invention will now be described more specifically with reference to the following embodiments. It is to be noted that the following descriptions of different embodiments of this invention are presented herein for purpose of illustration and description only, and it is not intended to limit the scope of the present invention.

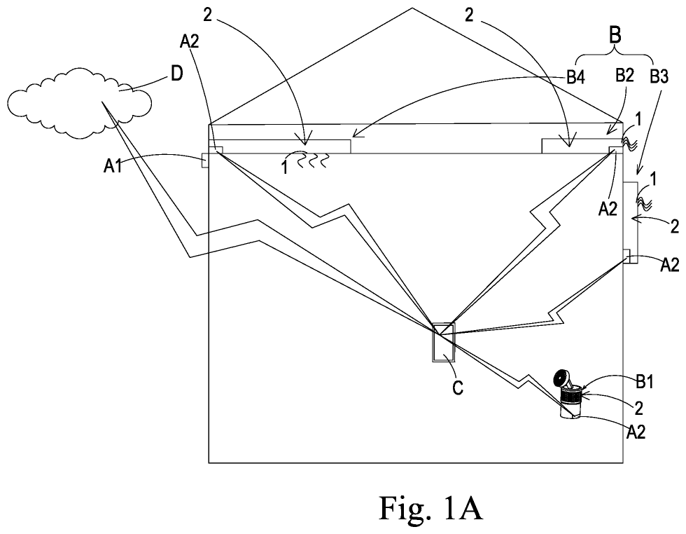

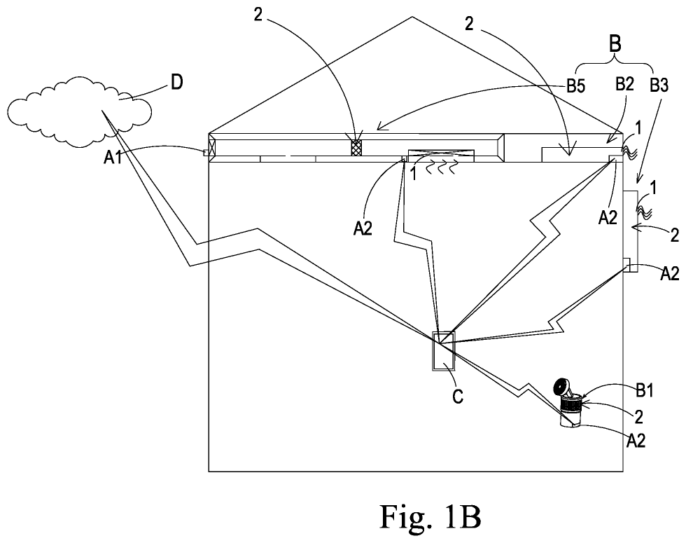

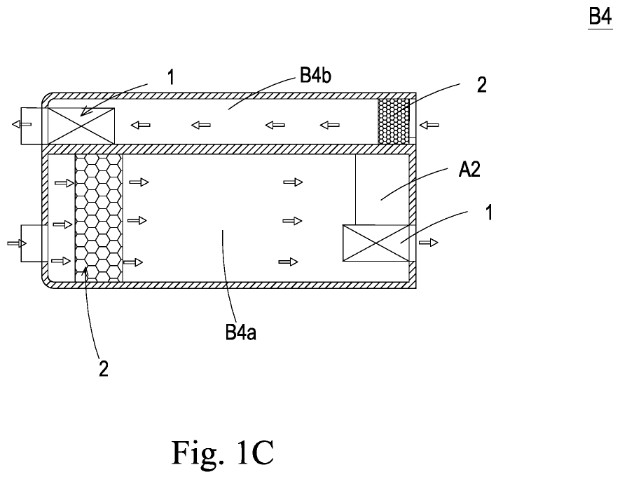

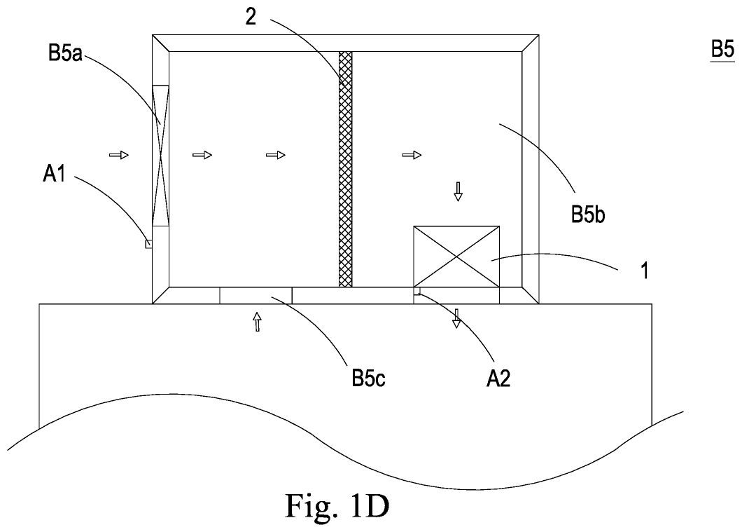

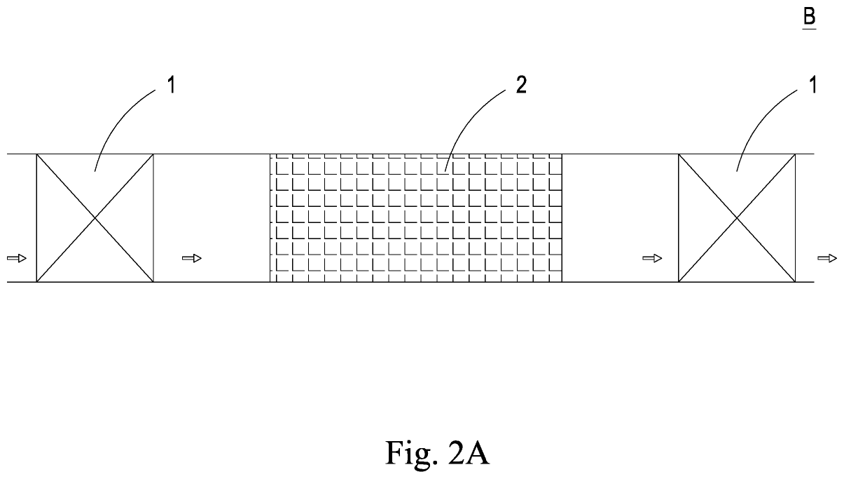

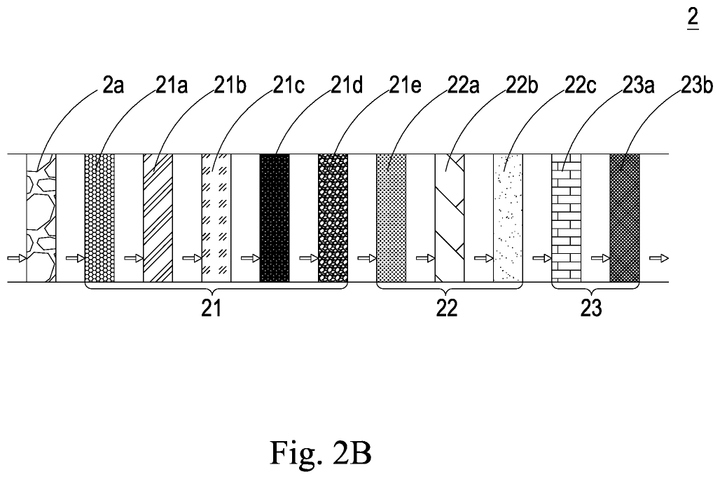

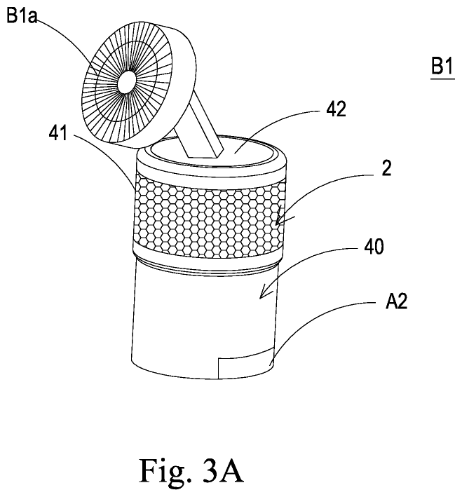

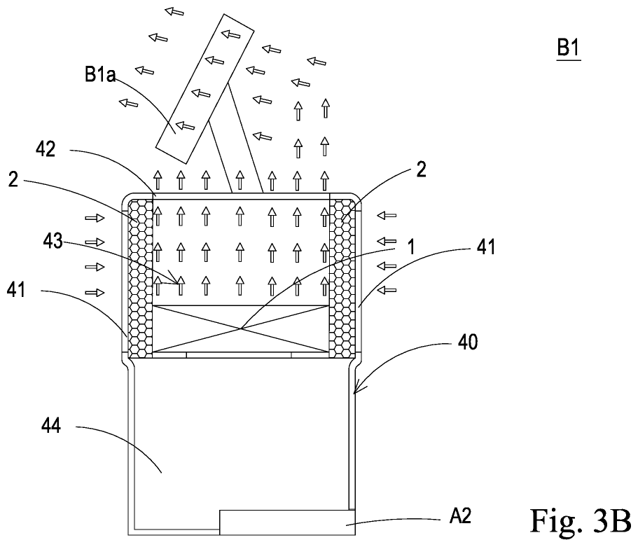

Please refer to FIG. 1A and FIG. 1B, FIG. 2A and FIG. 2B, and FIG. 3A and FIG. 3B, according to one or some embodiments of the present invention, a system for detecting and cleaning indoor air pollution includes at least one outdoor gas detection device A1, a plurality of indoor gas detection devices A2, a plurality of filtering devices B, and a control central processor C.

Accordingly, the at least one outdoor gas detection device A1 is configured to detect a qualitative property and a concentration of an air pollution of an outdoor gas and transmit an outdoor air pollution data. The indoor gas detection devices A2 are configured to detect a qualitative property and a concentration of an air pollution in the indoor space and output an indoor air pollution data. The control central processor C is configured to receive the outdoor air pollution data detected by the at least one outdoor gas detection device A1 and the indoor air pollution data detected by the indoor gas detection devices A2, perform an intelligent computation, namely, the artificial intelligent (AI) computation and big data comparison to determine an air pollution location in the indoor space and transmit a control command intelligently and selectively. For further definition of the air pollution (namely the polluted gas or polluted air) as mentioned in the above of embodiments, the air pollution may include at least one selected from the group consisting of particulate matters, carbon monoxide (CO), carbon dioxide (CO₂), ozone (O₃), sulfur dioxide (SO₂), nitrogen dioxide (NO₂), lead (Pb), total volatile organic compounds (TVOC), formaldehyde (HCHO), bacteria, fungi, viruses, and any combination thereof.

In some embodiments, in the intelligent computation, the control central processor C receives the outdoor air pollution data detected by the at least one outdoor gas detection device al and the indoor air pollution data detected by the indoor gas detection devices A2 by connecting to a cloud processing device D through a wireless transmission so as to perform artificial intelligent (AI) computation and big data comparison to locate the air pollution location in the indoor space and transmit the control command intelligently and selectively. According to one embodiment of the present invention, the control central processor C receives a highest data among the indoor air pollution data through the cloud processing device D to locate the air pollution location in the indoor space and transmit the control command intelligently and selectively in the intelligent computation. In another embodiment, the control central processor C receives the indoor air pollution data detected by at least three of the indoor gas detection devices A2 through the cloud processing device D to locate the air pollution location in the indoor space and transmit the control command intelligently and selectively due to the at least three of the indoor air pollution data in the intelligent computation.

As aforementioned, each of the filtering devices B comprises at least one blower B1 and at least one filtering component 1, also, each of the filtering devices B is provided with a corresponding one of the indoor gas detection devices A2. As shown in FIG. 1A, in some embodiments, the filtering devices B of the system for detecting and cleaning indoor air pollution may include a directing filtering device B1, a ventilator B2, a cooker hood B3, and a refresh air ventilation device B4. Alternatively, in some other embodiments, the filtering devices B of the system for detecting and cleaning indoor air pollution may include a directing filtering device B1, a ventilator B2, a cooker hood B3, and a heating, ventilation and air conditioning (HVAC) system B5. It should be noted that, the type or the number of the filtering devices B can be configured according to the volume of the indoor space, moreover, the indoor gas detection device A2 disposed on the filtering device B receives the control command to control the blower 1 to be driven in order to generate an air convection, therefore the circulative filtration and the rapid clean of the air pollution can be achieved by the filtering component 2, allowing the indoor air pollution data to approach to a non-detection state and the air in the indoor space to a safe and breathable state. In some embodiments, the safety detection value includes a detection value in which the air pollution data approaches to almost zero. Alternatively, in some embodiments, the safety detection value includes at least one selected from the group consisting of a concentration of PM2.5 which is less than 15 μg/m³, a concentration of carbon dioxide which is less than 1000 ppm, a concentration of total volatile organic compounds which is less than 0.56 ppm, a concentration of formaldehyde which is less than 0.08 ppm, a colony-forming unit per cubic meter of bacteria which is less than 1500 CFU/m³, a colony-forming unit per cubic meter of fungi which is less than 1000 CFU/m³, a concentration of sulfur dioxide which is less than 0.075 ppm, a concentration of nitrogen dioxide which is less than 0.1 ppm, a concentration of carbon monoxide which is less than 9 ppm, a concentration of ozone which is less than 0.06 ppm, a concentration of lead which is less than 0.15 μg/m³, and any combination thereof.

In one embodiment, as shown in FIG. 1A and FIG. 1B, the filtering devices B comprise at least one directing filtering device B1A directional blower B1a is on the at least one directing filtering device B1 and, the directional blower B1a is capable of being moved upwardly and downwardly, as well as rotating with respect to the at least one directing filtering device B1. The indoor gas detection device A2 of the at least one directing filtering device B1 receives the control command to enable the operation of the at least one directing filtering device B1 and control the directional blower B1a to be directed toward the air pollution location so as to generate a directed air convection, therefore the circulative filtration and the rapid clean of the air pollution by the at least one filtering component 2 can be achieved to allow the indoor air pollution data to be a safety detection value in which the air pollution data approaches to a non-detection state, and the gas (air) in the indoor space is cleaned to a safe and breathable state.

Moreover, as the system for detecting and cleaning indoor air pollution shown in FIG. 1A and FIG. 1C, the refresh air ventilation device B4 comprises an inlet channel B4a and a ventilation channel B4b, wherein at least one blower 1 and at least one filtering component 2 are disposed in the inlet channel B4a, and at least one blower 1 and at least one filtering component 2 are disposed in the ventilation channel B4b. When the control central processor C receives the outdoor air pollution data detected by the at least one outdoor gas detection device A1 and the indoor air pollution data detected by the indoor gas detection devices A2 to perform the intelligent computation, if the indoor air pollution data is greater than the outdoor air pollution data, the control central processor C transmits the control command to the indoor gas detection device A2 of the refresh air ventilation device B4 to enable the at least one blower 1 in the inlet channel B4a, guiding the outdoor gas into the inlet channel B4a and to enable the at least one filtering component 2 in the inlet channel B4a to filter the outdoor gas in order to allow the outdoor gas to enter the indoor space; meanwhile, the at least one blower 1 in the ventilation channel B4b is enabled to guide the indoor gas into the ventilation channel B4b, and the indoor gas is filtered by the at least one filtering component 2 in the ventilation channel B4b and discharged out of the indoor space. Therefore, when the system for detecting and cleaning indoor air pollution detects that the indoor air pollution data is greater than the outdoor air pollution data, the at least one blower 1 in the inlet channel B4a guide the outdoor gas into the inlet channel B4a and enable the at least one filtering component 2 in the inlet channel B4a to filter the outdoor gas so as to allow the outdoor gas to enter the indoor space, also the indoor gas is filtered by the at least one filtering component 2 in the ventilation channel B4b and discharged to the outdoor space, thus the air pollution in the indoor space is rapidly exchanged and discharged to the outdoor space, and the air pollution in the indoor space is filtered by the at least one filtering component 2 to allow the indoor air pollution data to be the safety detection value.

Furthermore, as the system for detecting and cleaning indoor air pollution shown in FIG. 1B and FIG. 1D, the heating, ventilation and air conditioning (HVAC) system comprises a gate Ba and a plurality of channels B5b. The gate B5a controls the channels B5b to introduce the outdoor gas. The channels B5b comprise at least one blower 1 and is connected with the indoor space. The gate B5a controls the outdoor gas to be guided into the channels B5b by the at least one blower 1 and filtered by the at least one filtering component 2, thus the outdoor gas is introduced into the indoor space. The channels B5b have a return inlet B5c adapted to introduce the indoor gas in the indoor space back into the channels B5b to make the circulative filtration. When the control central processor C receives the outdoor air pollution data detected by the at least one outdoor gas detection device A1 and the indoor air pollution data detected by the indoor gas detection devices A2 to perform the intelligent computation, if the indoor air pollution data is greater than the outdoor air pollution data, the control central processor C transmits the control command to the indoor gas detection device A2 of the HVAC system B5 to control the gate B5a to be opened and to enable the at least one blower 1, so that the air pollution in the indoor space is exchanged and discharged to the outdoor space. Therefore, when the system for detecting and cleaning indoor air pollution detects that the indoor air pollution data is greater than the outdoor air pollution data, the HVAC system B5 controls the gate B5a to be opened, then the air pollution in the indoor space is rapidly exchanged and discharged to the outdoor space, and the air pollution in the indoor space is filtered by the at least one filtering component 2, allowing the indoor air pollution data to be the safety detection value.

According to one or some embodiments of the present invention, as to the system for detecting and cleaning indoor air pollution, at least one outdoor gas detection device A1 and a plurality of indoor gas detection devices A2 are utilized to detect and compare the indoor gas and the outdoor gas in order to determine whether the air pollution in the indoor space would be exchanged and discharged to the outdoor space. Therefore, the air pollution in the indoor space is filtered by the at least one filtering component 2 to allow the indoor air pollution data to be the safety detection value. Moreover, in the embodiment of the system for detecting and cleaning indoor air pollution shown in FIG. 1A and FIG. 1B, the intelligent computation locates the air pollution location in the indoor space, the control command is intelligently and selectively transmitted to a filtering device B (for example, a directing filtering device B1) at the air pollution location and to rest of the filtering devices B (for example, the ventilator B2, the cooker hood B3, the refresh air ventilation device B4, or the HVAC system B5) which are outside the air pollution location respectively, enabling the operations of the filtering device B and the rest of the filtering devices B to generate the air convection directed to the air pollution. Hence, the air convection accelerates the filtering of the air pollution at the air pollution location and the air pollution outside the air pollution location which is diffused, moved, and directed by the air convection, and the filtering components 2 of the rest of the filtering devices B outside the air pollution location are enabled intelligently and selectively, therefore the air pollution in the indoor space is filtered to allow the indoor air pollution data to be the safety detection value in which the air pollution data approaches to a non-detection state, and the gas in the indoor space is cleaned to the safe and breathable state.

In other words as shown in FIG. 1A or FIG. 1B, after the indoor air pollution devices A2 of the filtering device B detect the air pollution, the intelligent computation determines the air pollution location in the indoor space. Accordingly, supposed that the air pollution location is at a peripheral region of the directing filtering device B1, the directional blower B1a of the directing filtering device B1 is controlled to be directed toward the air pollution location so as to generate a directed air convection, therefore the circulative filtration and the rapid clean of the air pollution by the filtering component 2 of the directing filtering device B1 can be achieved to allow the indoor air pollution data to be a safety detection value in which the air pollution data approaches to the a non-detection state, and the gas in the indoor space is cleaned to a safe and breathable state. In addition, supposed that the air pollution location is at a peripheral region of the cooker hood B3, the directing filtering device B1 intelligently guides directed air convection toward the peripheral region of the cooker hood B3. Therefore, the air pollution can be filtered not only by the filtering component 2 of the cooker hood B3, but also can be diffused, moved, and directed by the directed air convection toward the rest of the filtering devices B (for example, the ventilator B2, the refresh air ventilation device B4, or the HVAC system B5). Therefore, the air pollution in the indoor space is filtered by the filtering components 2 of the rest of the filtering devices B (for example, the ventilator B2, the refresh air ventilation device B4, or the HVAC system B5) to allow the indoor air pollution data to be the safety detection value in which the air pollution data approaches to the non-detection state, making the gas in the indoor space be cleaned to the safe and breathable state.

As aforementioned, the system for detecting and cleaning indoor air pollution of the present invention, at least one outdoor gas detection device A1 and a plurality of indoor gas detection devices A2 are utilized to detect and compare the indoor gas and the outdoor gas in order to determine whether the air pollution in the indoor space would be exchanged and discharged to the outdoor space. Moreover, through the detection of the indoor gas detection devices A2 in the filtering devices B (for example, the ventilator B2, the cooker hood B3, the refresh air ventilation device B4, or the HVAC system B5) of the indoor space, as well as the wireless transmission of the control central processor C, the intelligent computation is performed to locate the air pollution location in the indoor space, and the control command is transmitted intelligently and selectively to enable the filtering devices B (for example, the ventilator B2, the cooker hood B3, the refresh air ventilation device B4, or the HVAC system B5) to be driven to generate a certain directed air convection, thereby the circulative filtration and the rapid clean of the air pollution can be achieved by the filtering component 2, allowing the indoor air pollution data to be a safety detection value in which the air pollution data approaches to the non-detection state, making the gas in the indoor space be cleaned to a safe and breathable state.

The following descriptions are provided to understand how the system for detecting and cleaning indoor air pollution to allow the indoor air pollution data to be a safety detection value in which the air pollution data approaches to the non-detection state and to allow the air in the indoor space to be cleaned to a safe and breathable state. Please refer to FIG. 2A and FIG. 2B, the filtering component 2 of the filtering device B may be a physical-type or chemical-typed filtering device. In one or some embodiments, the filtering component 2 of the filtering device B is a physical-typed filtering device, the filtering component 2 filters the air pollution physically by a filter to block and absorb the air pollution. In some embodiments, the filter is a high-efficiency particulate air filter 2a for absorbing the chemical smog, bacteria, dusts, particles, and pollens contained in the polluted gas, thereby the polluted gas introduced into the system can be filtered and purified. In one or some embodiments, the filtering component 2 of the filtering device B is a chemical-typed filtering device, the filtering component 2 filters the air pollution chemically by applying a degradation layer 21 on the filtering component 2. In some embodiments, the degradation layer 21 may be an activated carbon 21a for filtering organic and inorganic substances and for filtering colored or odor substances. In some embodiments, the degradation layer 21 may be a cleansing factor layer 21b having chlorine dioxide for suppressing viruses, bacteria, fungus, influenza A virus, influenza B virus, Enterovirus, and Norovirus in the polluted gas introduced into the system. Accordingly, the suppressing rate may exceed 99%, allowing the reduction of the cross infections of the viruses. In some embodiments, the degradation layer 21 may be an herbal protection coating layer 21c including the extracts of Rhus chinensis Mill (may be Rhus chinensis Mill from Japan) and the extracts of Ginkgo biloba to efficiently perform anti-allergy function and destroy cell surface proteins of influenza viruses (e.g., influenza virus subtype H1N1). In some embodiments, the degradation layer 21 may be a layer of silver ions 21d for suppressing viruses, bacteria, and fungus in the polluted gas introduced into the system. In some embodiments, the degradation layer 21 may be a zeolite mesh 21e for removing ammonia, heavy metals, organic pollutants, Escherichia coli, phenol, chloroform, or anion surfactants. In some embodiments, the filtering component 2 filters the air pollution chemically along with a light illumination 22. In some embodiments, the light illumination 22 is a photocatalyst unit including a photocatalyst 22a and an ultraviolet light 22b. When the photocatalyst 22a is illuminated by the ultraviolet light 22b, the light energy is converted into electrical energy in order to degrade the hazardous matters in the polluted gas to achieve the effect of filtration and purification. In some embodiments, the light illumination 22 is a photo plasma unit including a nanometer light tube 22c. The introduced polluted gas is illuminated by the nanometer light tube, making the oxygen molecules and water molecules in the polluted gas decompose into photo plasma with high oxidative power for generating a plasma flow which is capable of destroying the organic molecules. Accordingly, volatile organic compounds (VOC) such as formaldehyde and toluene in the polluted gas can be decomposed into water and carbon dioxide. In some embodiments, the filtering component 2 filters the air pollution chemically along with a degradation unit 23. In some embodiments, the degradation unit 23 is a negative ion unit 23a; through applying high voltage discharging to the introduced polluted gas, the particulates carry with positive charges in the polluted gas are adhered to the negative charges on the negative ion unit 23a. In some embodiments, the degradation unit 23 is a plasma ion unit 23b; when the polluted gas is introduced into the system, the oxygen molecules and the water molecules in the polluted gas are ionized to generate cations (H⁺) and anions (O₂⁻). After the substances attached with water molecules around the ions attach on the surfaces of viruses and bacteria, the water molecules will be converted into oxidative oxygen ions (hydroxyl ions, OH⁻ ions) with high oxidative power under chemical reaction, resulting in the oxidative oxygen ions take away the hydrogen ions of the proteins on the surfaces of the viruses and the bacteria so as to oxidize and decompose the viruses and bacteria. Therefore, the introduced gas can be filtered.

According to the descriptions stated as above, each of the filtering devices B includes at least one blower 1 and at least one filtering component 2, wherein the blower 1 has the ability of transmitting the air bi-directionally, including the extraction and ejection. In this embodiment, the arrow shown in the figures indicates the direction of the air flow. The blower 1 may be disposed in front of the filtering component 2 or behind the filtering component 2, also the blowers 1 may be disposed in front of and behind the filtering component 2 (as shown in FIG. 2A) simultaneously. Accordingly, the blower 1 can be adjusted and modified according to any practical scenario by the person in the art.

Moreover, as shown in FIG. 3A and FIG. 3B, according to one or some embodiments of the present invention, the directing filtering device 1 is referred to a filtering device that can generate a directed air convection intelligently, and the directing filtering device 1 includes a blower 1, a directional blower 1a, a filtering component 2, a main body 40, an inlet opening 41, an outlet opening 42, a gas passage 43, a power device 44, and an indoor gas detection device A2. The inlet opening 41, the outlet opening 42, and the gas passage 43 are disposed in the main body 40, the gas passage 43 is disposed between the inlet opening 41 and the outlet opening 42, and the filtering component 2 is disposed in the gas passage 43 to filter the air pollution guided into the gas passage 43. The blower 1 is disposed in the gas passage 43 and at the central portion of the filtering component 2 to guide the air pollution into the filtering component 2 from the inlet opening 41 to outlet opening 42 for filtration and purification. The power device 44 is electrically connected to the blower 1, the directional blower 1a, and the indoor gas detection device A2 for providing the operation power for these components. Alternatively, in some embodiments, the power device 44 may be connected to the supply mains. The directional blower B1a is configured on the main body 40 of the directing filtering device B1 to guide the air convection, and the directional blower B1a is capable of being moved upwardly and downwardly, as well as rotating with respect to the directing filtering device B1.

In addition, please refer to FIG. 1A and FIG. 1B, the outdoor gas detection device A1 is disposed in the outdoor space and configured to detect the qualitative property and the concentration of the air pollution of the outdoor gas, further, the indoor gas detection devices A2 are disposed in the indoor space and configured to detect the qualitative property and the concentration of the air pollution of the indoor gas. To illustrate the embodiments of the present invention clearly, the detail structures of the outdoor gas detection device A1 and the indoor gas detection device A2 are illustrated as below.

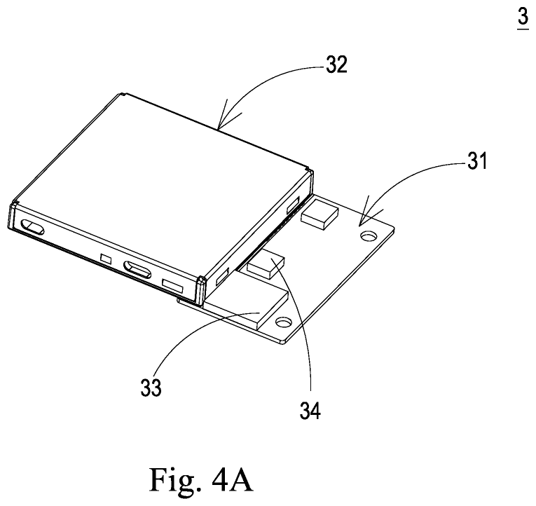

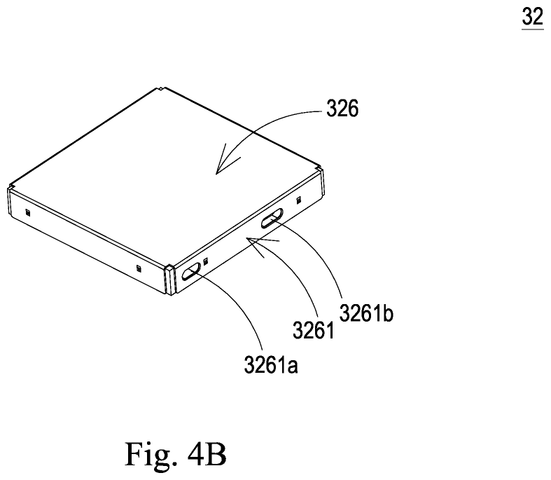

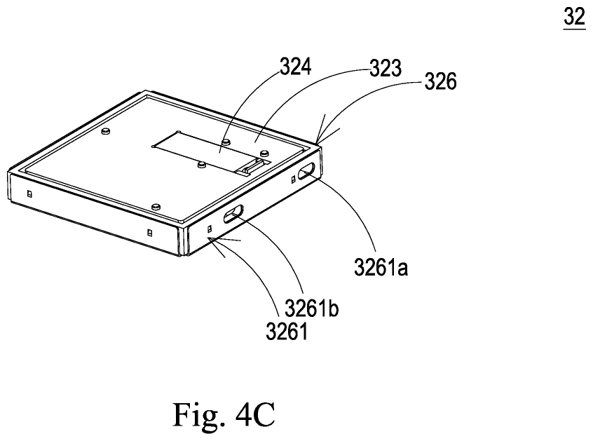

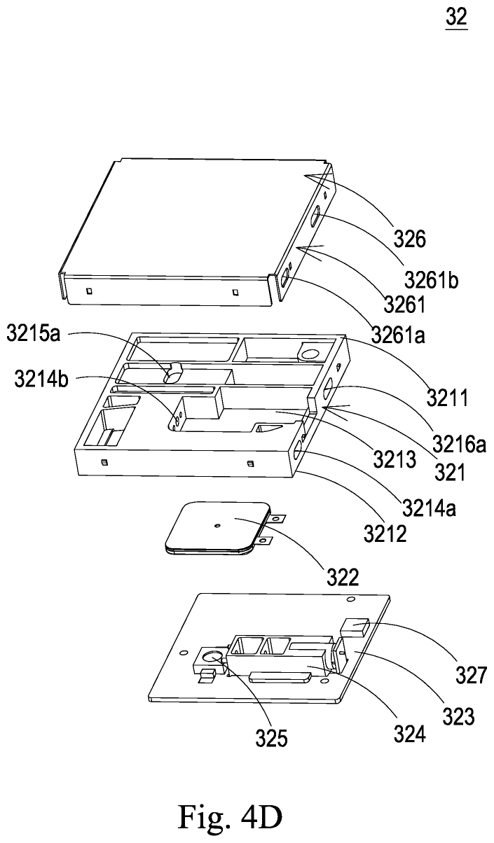

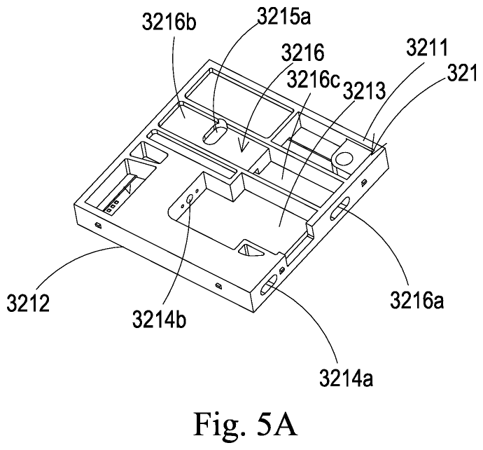

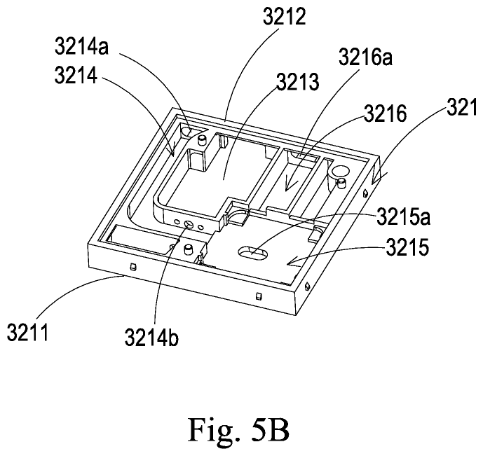

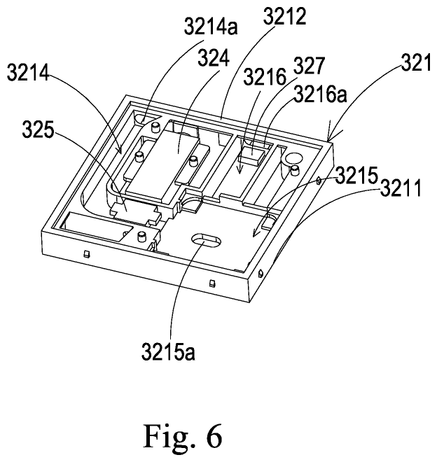

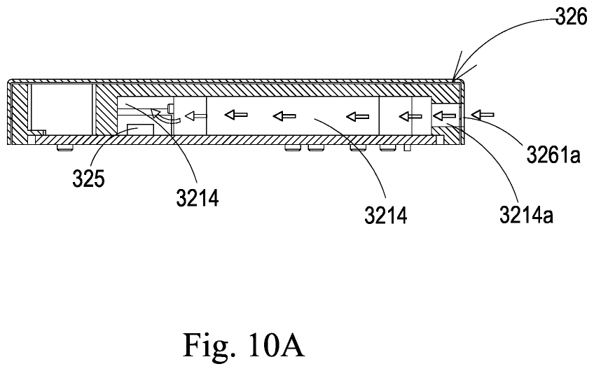

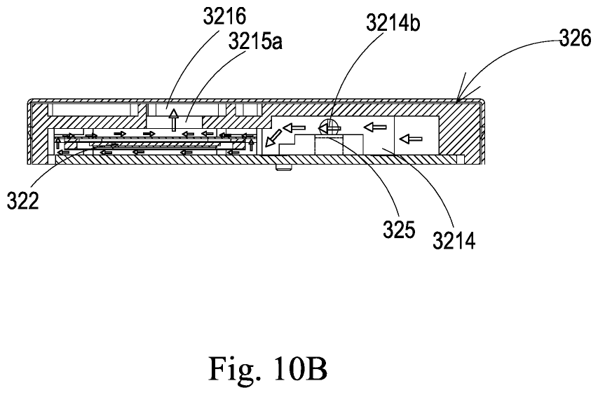

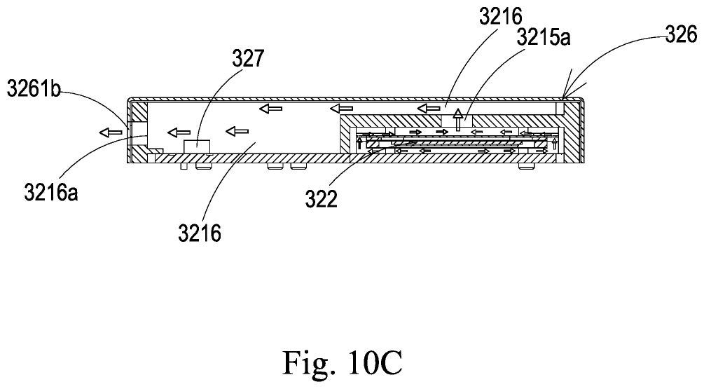

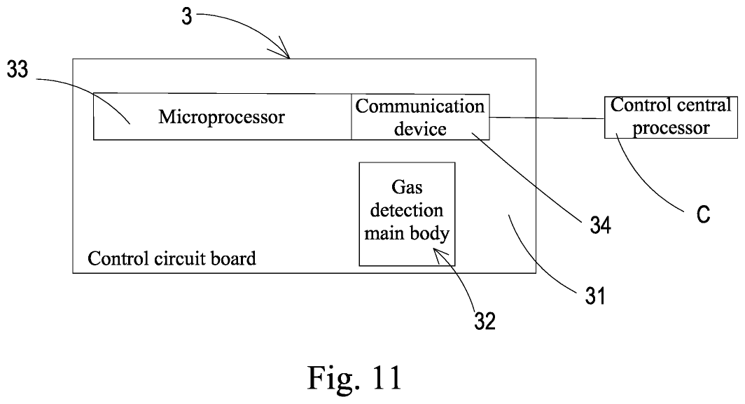

According to one or some embodiments of the present invention, each of the outdoor gas detection device A1 and the indoor gas detection device A2 is a gas detection device, which would be indicated by reference number 3 in the descriptions below. Please refer to FIG. 4A to FIG. 11. The gas detection device 3 includes a control circuit board 31, a gas detection main body 32, a microprocessor 33, and a communication device 34. The gas detection main body 32, the microprocessor 33, and the communication device 34 are integrally packaged with the control circuit board 31 and electrically connected to each other. The microprocessor 33 and the communication device 34 are disposed on the control circuit board 31, and the microprocessor 33 controls a driving signal of the gas detection main body 32 to enable the operation of the gas detection main body 32, so that the gas detection main body 32 detects the air pollution and outputs a detection signal, and the microprocessor 33 receives the detection signal so as to compute, process, and output the air pollution data, therefore the microprocessor 33 provides the communication device 34 with the air pollution data for wirelessly transmitting outwardly to the control central processor C (as shown in FIG. 11). The wireless communication is implemented by using a Wi-Fi module, a Bluetooth module, a radiofrequency identification (RFID) module, or a near field communication module.

Please refer to FIG. 4A to FIG. 9A. In one or some embodiments, the gas detection main body 32 includes a base 321, a piezoelectric actuator 322, a driving circuit board 323, a laser component 324, a particulate sensor 325, and an outer cover 326. The base 321 has a first surface 3211, a second surface 3212, a laser installation region 3213, a gas inlet groove 3214, a gas-guiding component installation region 3215, and a gas outlet groove 3216. The first surface 3211 and the second surface 3212 are opposite to each other. The laser installation region 3213 is formed by hollowing out the base 321 from the first surface 3211 to the second surface 3212 for accommodating the laser component 324. The outer cover 326 covers the base 321 and has a side plate 3261. The side plate 3261 has a gas inlet opening 3261a and a gas outlet opening 3261b. The gas inlet groove 3214 is recessed from the second surface 3212 and located adjacent to the laser installation region 3213. The gas inlet groove 3214 has a gas inlet through hole 3214a and two lateral walls. The gas inlet through hole 3214a is in communication with the outside environment of the base 321 and is corresponding to the gas inlet opening 3261a of the outer cover 326. Two light penetration windows 3214b penetrate the two lateral walls of the gas inlet groove 3214 and are in communication with the laser installation region 3213. Therefore, when the first surface 3211 of the base 321 is covered by the outer cover 326, and the second surface 3212 of the base 321 is covered by the driving circuit board 323, a gas inlet path can be defined by the gas inlet groove 3214.

The gas-guiding component installation region 3215 is recessed from the second surface 3212 and in communication with the gas inlet groove 3214. A ventilation hole 3215a penetrates a bottom surface of the gas-guiding component installation region 3215. Each of the four corners of the gas-guiding component installation region 3215 has a positioning bump 3215b. The gas outlet groove 3216 has a gas outlet through hole 3216a, and the gas outlet through hole 3216a is corresponding to the gas outlet opening 3261b of the outer cover 326. The gas outlet groove 3216 includes a first region 3216b and a second region 3216c. The first region 3216b is recessed from a portion of the first surface 3211 corresponding to a vertical projection region of the gas-guiding component installation region 3215. The second region 3216c is at a portion extending from a region that is not corresponding to the vertical projection region of the gas-guiding component installation region 3215, and the second region 3216c is hollowed out from the first surface 3211 to the second surface 3212. The first region 3216b is connected to the second region 3216c to form a stepped structure. Moreover, the first region 3216b of the gas outlet groove 3216 is in communication with the ventilation hole 3215a of the gas-guiding component installation region 3215, and the second region 3216c of the gas outlet groove 3216 is in communication with the gas outlet through hole 3216a. Therefore, when the first surface 3211 of the base 321 is covered by the outer cover 326 and the second surface 3212 of the base 321 is covered by the driving circuit board 323, a gas outlet path can be defined by the gas outlet groove 3216 and the driving circuit board 323.

Furthermore, the laser component 324 and the particulate sensor 325 are disposed on the driving circuit board 323 and located in the base 321. The laser component 324 and the particulate sensor 325 are electrically connected to the driving circuit board 323. It should notice that the driving circuit board 323 is omitted to clearly explain the positions of the laser component 324, the particulate sensor 325, and the base 321. In the embodiment of the present invention, the laser component 324 is located at the laser installation region 3213 of the base 321. The particulate sensor 325 is located at the gas inlet groove 3214 of the base 321 and aligned with the laser component 324. Moreover, the laser component 324 is corresponding to the light penetration windows 3214b so as to allow the light beam emitted by the laser component 324 to pass therethrough and into the gas inlet groove 3214. The light path of the light beam emitted by the laser component 324 passes through the light penetration windows 3214b and is orthogonal to the gas inlet groove 3214. The light beam emitted by the laser component 324 passes into the gas inlet groove 3214 through the light penetration windows 3214b, thereby the particulate matters in the gas inlet groove 3214 is illuminated by the light beam. When the light beam contacts the gas, the light beam will be scattered and generate light spots. Hence, the light spots generated by the scattering are received and calculated by the particulate sensor 325 located at the position orthogonal to the gas inlet groove 3214 to obtain the detection data of the gas. Furthermore, a gas sensor 327 is disposed on the driving circuit board 323 and is located at the gas outlet groove 3216 for detecting the polluted gas introduced into the gas outlet groove 3216, and the gas sensor 327 is electrically connected to the driving circuit board 323. In one embodiment of the present invention, the gas sensor 327 includes at least one selected from the group consisting of a volatile organic compound detector capable of detecting gas information of carbon dioxide (CO₂) or total volatile organic compounds (TVOC), a formaldehyde sensor capable of detecting gas information of formaldehyde (HCHO) gas, a bacterial sensor capable of detecting information of bacteria or fungi, and a virus sensor capable of detecting information of viruses, and any combination thereof.

Moreover, the piezoelectric actuator 322 is located at the square-shaped gas-guiding component installation region 3215 of the base 321, and the gas-guiding component installation region 3215 is in communication with the gas inlet groove 3214. When the piezoelectric actuator 322 is enabled, the gas in the gas inlet groove 3214 is inhaled into the piezoelectric actuator 322, passing through the ventilation hole 3215a of the gas-guiding component installation region 3215, and entering the gas outlet groove 3216. Moreover, the driving circuit board 323 covers the second surface 3212 of the base 321. The laser component 324 and the particulate sensor 325 are disposed on the driving circuit board 323 and electrically connected to the driving circuit board 323. As the outer cover 326 covers the base 321, the gas inlet opening 3261a is corresponding to the gas inlet through hole 3214a of the base 321, and the gas outlet opening 3216b is corresponding to the gas outlet through hole 3216a of the base 321.

Furthermore, the piezoelectric actuator 322 includes a nozzle plate 3221, a chamber frame 3222, an actuation body 3223, an insulation frame 3224, and a conductive frame 3225. The nozzle plate 3221 is made by a flexible material and has a suspension sheet 3221a and a hollow hole 3221b. The suspension sheet 3221a is a flexible sheet which can bend and vibrate. The shape and the size of the suspension sheet 3221a approximately corresponding to the inner edge of the gas-guiding component installation region 3215. The hollow hole 3221b penetrates through the center portion of the suspension sheet 3221a for the gas flowing therethrough. In one embodiment of the present invention, the shape of the suspension sheet 3221a can be selected from square, circle, ellipse, triangle, or polygon.

Furthermore, the chamber frame 3222 is stacked on the nozzle plate 3221, and the shape of the chamber frame 3222 is corresponding to the shape of the nozzle plate 3221. The actuation body 3223 is stacked on the chamber frame 3222. A resonance chamber 3226 is collectively defined between the actuation body 3223, the chamber frame 3222, and the suspension sheet 3221a. The insulation frame 3224 is stacked on the actuation body 3223. The appearance of the insulation frame 3224 is similar to the appearance of the chamber frame 3222. The conductive frame 3225 is stacked on the insulation frame 3224. The appearance of the conductive frame 3225 is similar to the appearance of the insulation frame 3224. The conductive frame 3225 has a conductive pin 3225a and a conductive electrode 3225b. The conductive pin 3225a extends outwardly from the outer edge of the conductive frame 3225, and the conductive electrode 3225b extends inwardly from the inner edge of the conductive frame 3225. Moreover, the actuation body 3223 further includes a piezoelectric carrying plate 3223a, an adjusting resonance plate 3223b, and a piezoelectric plate 3223c. The piezoelectric carrying plate 3223a is stacked on the chamber frame 3222, and the adjusting resonance plate 3223b is stacked on the piezoelectric carrying plate 3223a. The piezoelectric plate 3223c is stacked on the adjusting resonance plate 3223b. The adjusting resonance plate 3223b and the piezoelectric plate 3223c are accommodated in the insulation frame 3224. The conductive electrode 3225b of the conductive frame 3225 is electrically connected to the piezoelectric plate 3223c. In one preferred embodiment of the present invention, the piezoelectric carrying plate 3223a and the adjusting resonance plate 3223b are both made of conductive material(s). The piezoelectric carrying plate 3223a has a piezoelectric pin 3223d. The piezoelectric pin 3223d and the conductive pin 3225a are in electrical connection with a driving circuit (not shown) of the driving circuit board 323 to receive a driving signal (which may be a driving frequency and a driving voltage). The piezoelectric pin 3223d, the piezoelectric carrying plate 3223a, the adjusting resonance plate 3223b, the piezoelectric plate 3223c, the conductive electrode 3225b, the conductive frame 3225, and the conductive pin 3225a may together generate an electrical circuit for transmitting the driving signal, and the insulation frame 3224 is provided for electrically insulating the conductive frame 3225 from the actuation body 3223 to avoid short circuit, thereby the driving signal can be transmitted to the piezoelectric plate 3223c. When the piezoelectric plate 3223c receives the driving signal, the piezoelectric plate 3223c deforms owing to the piezoelectric effect, and thus the piezoelectric carrying plate 3223a and the adjusting resonance plate 3223b are driven to vibrate in a reciprocating manner.

Moreover, the adjusting resonance plate 3223b is disposed between the piezoelectric plate 3223c and the piezoelectric carrying plate 3223a as a cushion element so as to adjust the vibration frequency of the piezoelectric carrying plate 3223a. Generally, the thickness of the adjusting resonance plate 3223b is greater than the thickness of the piezoelectric carrying plate 3223a. The thickness of the adjusting resonance plate 3223b may be modified to adjust the vibration frequency of the actuation body 3223.

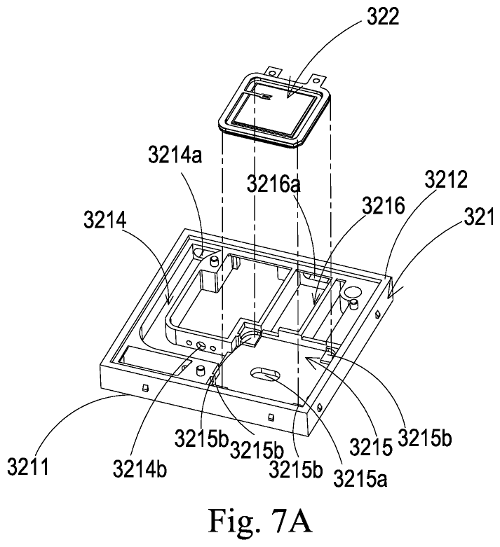

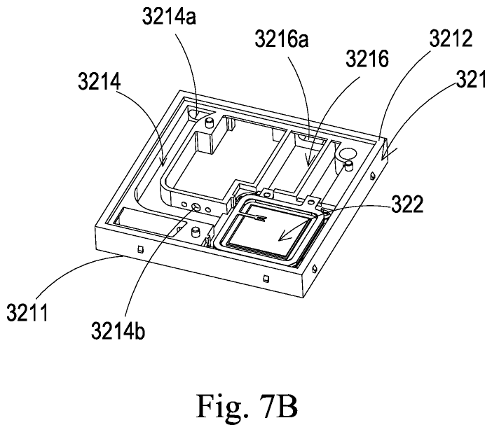

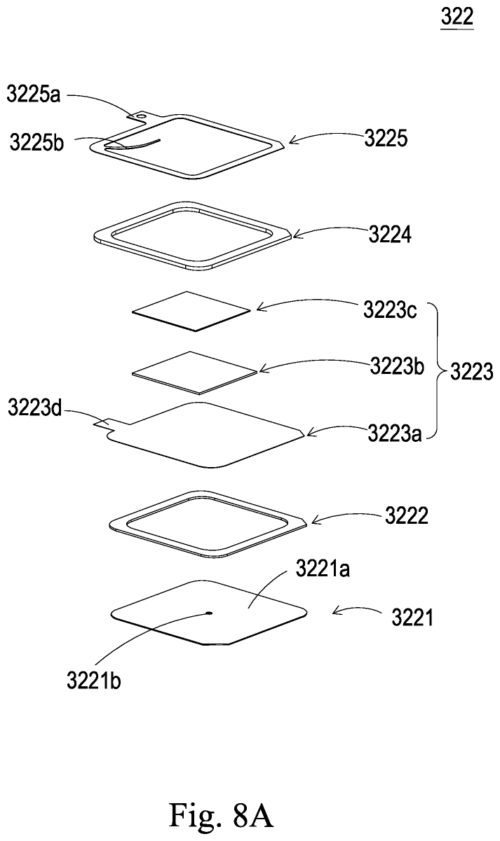

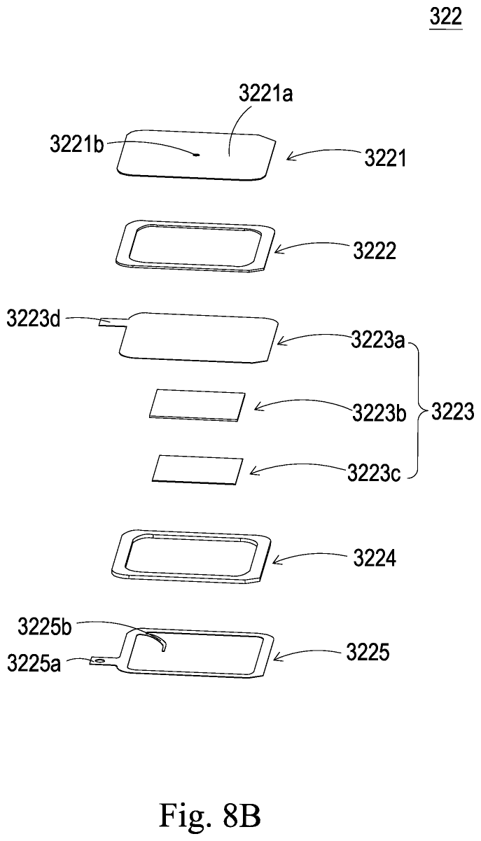

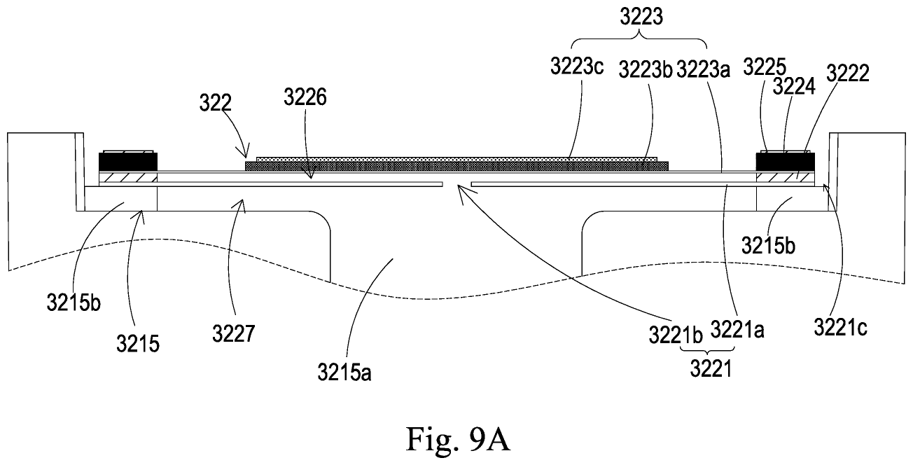

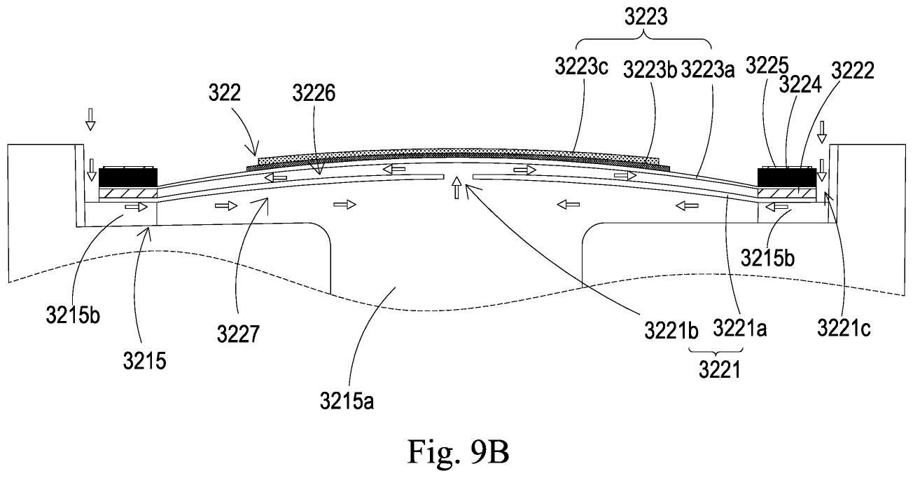

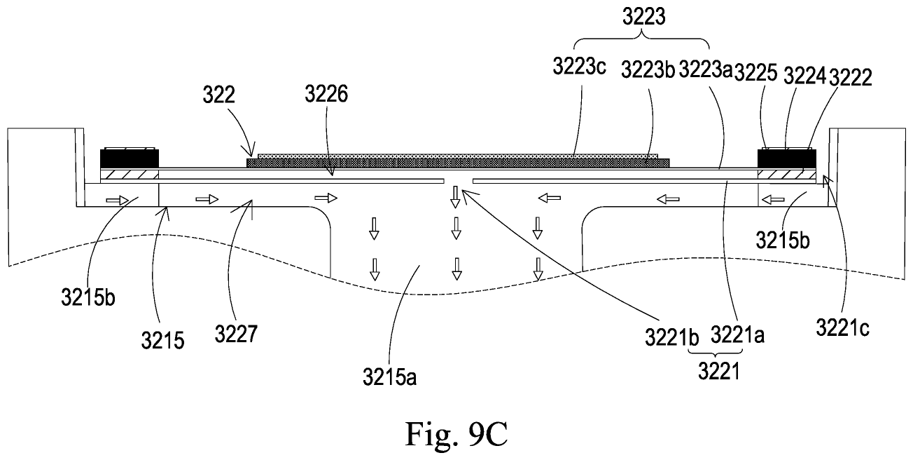

Please refer to FIG. 7A, FIG. 7B, FIG. 8A, FIG. 8B, and FIG. 9A. The nozzle plate 3221, the chamber frame 3222, the actuation body 3223, the insulation frame 3224, and the conductive frame 3225 are sequentially stacked and assembled and are positioned in the gas-guiding component installation region 3215, thereby a clearance 3221c is defined between the suspension sheet 3221a and the inner edge of the gas-guiding component installation region 3215 for the gas to pass therethrough. A gas flow chamber 3227 is formed between the nozzle plate 3221 and the bottom surface of the gas-guiding component installation region 3215. The gas flow chamber 3227 is in communication with the resonance chamber 3226 formed between the actuation body 3223, the chamber frame 3222, and the suspension sheet 3221a through the hollow hole 3221b of the nozzle plate 3221. In one aspect of the present invention, the resonance chamber 3226 and the suspension sheet 3221a can generate the Helmholtz resonance effect to improve the transmission efficiency of the gas through controlling the vibration frequency of the gas in the resonance chamber 3226 to be close to the vibration frequency of the suspension sheet 3221a. When the piezoelectric plate 3223c moves in a direction away from the bottom surface of the gas-guiding component installation region 3215, the piezoelectric plate 3223c drives the suspension sheet 3221a of the nozzle plate 3221 to move in the direction away from the bottom surface of the gas-guiding component installation region 3215 correspondingly. Hence, the volume of the gas flow chamber 3227 expands dramatically, therefore the internal pressure of the gas flow chamber 3227 decreases and creates a negative pressure, drawing the gas outside the piezoelectric actuator 322 to flow into the piezoelectric actuator 322 through the clearance 3221c and enter the resonance chamber 3226 through the hollow hole 3221b, thereby increasing the gas pressure of the resonance chamber 3226 and thus generating a pressure gradient. When the piezoelectric plate 3223c drives the suspension sheet 3221a of the nozzle plate 3221 to move toward the bottom surface of the gas-guiding component installation region 3215, the gas inside the resonance chamber 3226 is pushed to flow out quickly through the hollow hole 3221b to further push the gas inside the gas flow chamber 3227, thereby the converged gas can be quickly and massively ejected out of the gas flow chamber 3227 through the ventilation hole 3215a of the gas-guiding component installation region 3215 in a state closing to an ideal gas state under the Bernoulli's law.

Therefore, through repeating the steps as shown in FIG. 9B and FIG. 9C, the piezoelectric plate 3223c can bend and vibrate in a reciprocating manner. Further, after the gas is discharged out of the resonance chamber 3226, the internal pressure of the resonance chamber 3226 is lower than the equilibrium pressure due to the inertia, as a result, the pressure difference guides the gas outside the resonance chamber 3226 into the resonance chamber 3226 again. Therefore, through controlling the vibration frequency of the gas in the resonance chamber 3226 to be close to the vibration frequency of the piezoelectric plate 3223c, the resonance chamber 3226 and the piezoelectric plate 3223c can generate the Helmholtz resonance effect so as to achieve effective, high-speed, and large-volume gas transmission of the gas. Moreover, the gas enters the gas detection main body 32 from the gas inlet opening 3261a of the outer cover 326, flows into the gas inlet groove 3214 of the base 321 through the gas inlet through hole 3214a, and reaches the position of the particulate sensor 325. Furthermore, the piezoelectric actuator 322 continuously drives the gas into the gas inlet path so as to facilitate the gas inside the detection main body 32 to stably and quickly pass through the particulate sensor 325. Next, the light beam emitted by the laser component 324 passes through the light penetration windows 3214b, enters the gas inlet groove 3214, and illuminates the gas in the gas inlet groove 3214 which passes through the particulate sensor 325. When the light beam from the particulate sensor 325 illuminates on the particulate matters in the gas, the light beam will be scattered and generate light spots. The particulate sensor 325 receives and calculates the light spots generated by the scattering to obtain the information of the particulate matters in the gas such as the particle size and the number of the particulate matters. Moreover, the gas passing through the particulate sensor 325 is continuously introduced into the ventilation hole 3215a of the gas-guiding component installation region 3215 by the piezoelectric actuator 322 and enters the gas outlet groove 3216. Finally, after the gas enters the gas outlet groove 3216, since the piezoelectric actuator 322 continuously delivers the gas into gas outlet groove 3216, therefore the gas is continuously pushed and discharged out of the gas detection main body 32 through the gas outlet through hole 3216a and the gas outlet opening 3261b.

In some embodiments, the gas detection device 3 (which may be the outdoor gas detection device A1 or the indoor gas detection device A2) not only can detect the particulate matters in the gas, but also can obtain the property of the gas introduced into the gas detection device 3. For example, the gas may be formaldehyde, ammonia, carbon monoxide, carbon dioxide, oxygen, ozone, or the like. Therefore, the gas detection device 3 further includes a gas sensor 327. The gas sensor 327 is disposed on the driving circuit board 323 and is located at the gas outlet groove 3216 for detecting the polluted gas introduced into the gas outlet groove 3216, and the gas sensor 327 is electrically connected to the driving circuit board 323. Therefore, the gas sensor 327 can obtain the concentration or the property of the volatile organic compounds contained in the gas from the gas outlet path.

As above, one or some embodiments of the present invention provides a system for detecting and cleaning indoor air pollution, which not only can be utilized in an indoor space with a refresh air ventilation device but also can be utilized in an indoor space with an HVAC system. At least one outdoor gas detection device and a plurality of gas detection devices are utilized to detect and compare the indoor gas and the outdoor gas so as to determine whether the air pollution in the indoor space is to be exchanged and discharged to the outdoor space. Moreover, through the detection of the indoor gas detection devices in the filtering devices (for example, the ventilator, the cooker hood, the refresh air ventilation device, or the HVAC system) in the indoor space and the wireless transmission of the control central processor, the intelligent computation is performed to figure out the air pollution location in the indoor space, and the control command is transmitted intelligently and selectively to enable the filtering devices (for example, the ventilator, the cooker hood, the refresh air ventilation device, or the HVAC system) to be driven to generate a certain directed air convection, so that the air pollution can be repeatedly filtered and cleaned quickly by the filtering component to allow the indoor air pollution data to be a safety detection value in which the air pollution data approaches to almost zero, and the gas in the indoor space is cleaned to a safe and breathable state. Therefore, the air pollution in the indoor space can be filtered and cleaned instantly. Hence, a performance of locating the air pollution, guiding the air pollution, and cleaning and filtering the air pollution can be achieved.

The foregoing outlines features of several embodiments so that those skilled in the art may better understand the aspects of the present invention. Those skilled in the art should appreciate that they may readily use the present invention as a basis for designing or modifying other processes and structures for carrying out the same purposes and/or achieving the same advantages of the embodiments introduced herein. Those skilled in the art should also realize that such equivalent constructions do not depart from the spirit and scope of the present invention, and that they may make various changes, substitutions, and alterations herein without departing from the spirit and scope of the present invention.

What is claimed is:

1. A system for detecting and cleaning indoor air pollution comprising:
at least one outdoor gas detection device configured to detect a qualitative property and a concentration of an air pollution of an outdoor gas and output an outdoor air pollution data;
a plurality of indoor gas detection devices disposed in an indoor space and configured to detect a qualitative property and a concentration of an air pollution of an indoor gas and output an indoor air pollution data;
a control central processor, wherein the control central processor is configured to receive the outdoor air pollution data detected by the at least one outdoor gas detection device and the indoor air pollution data detected by the indoor gas detection devices, perform an intelligent computation to locate an air pollution location in the indoor space, and transmit a control command intelligently and selectively; and
a plurality of filtering devices, wherein each of the filtering devices comprises at least one blower and at least one filtering component, each of the filtering devices is provided with a corresponding one of the indoor gas detection devices wherein the indoor gas detection device disposed on each of the filtering device receives the control command to control the at least one blower to be driven to generate an air convection, and the air pollution in the indoor space is filtered by the at least one filtering component, wherein the filtering devices further comprise at least one directing filtering device;
wherein a directional blower is on the at least one directing filtering device, and the directional blower is capable of being moved upwardly and downwardly, as well as rotating with respect to the at least one directing filtering device;
wherein the indoor gas detection device disposed on the at least one directing filtering device receives the control command to enable the at least one directing filtering device and control the directional blower to be directed toward the air pollution location so as to generate a directed air convection, thereby the circulative filtration and the rapid clean of the air pollution by the at least one filtering component of the at least one directing filtering device can be achieved to allow the indoor air pollution data to be a safety detection value in which the air pollution data approaches to a non-detection state, and the gas in the indoor space is cleaned to a safe and breathable state.

2. The system for detecting and cleaning indoor air pollution according to claim 1, wherein the air pollution comprises at least one selected from the group consisting of particulate matters, carbon monoxide, carbon dioxide, ozone, sulfur dioxide, nitrogen dioxide, lead, total volatile organic compounds, formaldehyde, bacteria, fungi, viruses, and any combination thereof.

3. The system for detecting and cleaning indoor air pollution according to claim 1, wherein the safety detection value comprises a detection value in which the air pollution data approaches to almost zero.

4. The system for detecting and cleaning indoor air pollution according to claim 1, wherein the safety detection value includes at least one selected from the group consisting of a concentration of PM2.5 which is less than 15 μg/m³, a concentration of carbon dioxide (CO₂) which is less than 1000 ppm, a concentration of total volatile organic compounds (TVOC) which is less than 0.56 ppm, a concentration of formaldehyde (HCHO) which is less than 0.08 ppm, a colony-forming unit per cubic meter of bacteria which is less than 1500 CFU/m³, a colony-forming unit per cubic meter of fungi which is less than 1000 CFU/m³, a concentration of sulfur dioxide which is less than 0.075 ppm, a concentration of nitrogen dioxide which is less than 0.1 ppm, a concentration of carbon monoxide which is less than 9 ppm, a concentration of ozone which is less than 0.06 ppm, a concentration of lead which is less than 0.15 μg/m³, and any combination thereof.

5. The system for detecting and cleaning indoor air pollution according to claim 1, wherein the filtering devices further comprise at least one selected from a ventilator, a cooker hood, a refresh air ventilation device, and any combination thereof.

6. The system for detecting and cleaning indoor air pollution according to claim 5, wherein the refresh air ventilation device comprises an inlet channel and a ventilation channel, at least one blower and at least one filtering component are disposed in the inlet channel, and at least one blower and at least one filtering component are disposed in the ventilation channel; wherein the control central processor receives the outdoor air pollution data detected by the at least one outdoor gas detection device and the indoor air pollution data detected by the indoor gas detection devices to perform the intelligent computation; if the indoor air pollution data is greater than the outdoor air pollution data, the control central processor transmits the control command to the indoor gas detection device of the refresh air ventilation device to enable the at least one blower in the inlet channel to guide the outdoor gas into the inlet channel and the at least one filtering component in the inlet channel to filter the outdoor gas so as to allow the outdoor gas to enter the indoor space, and the indoor gas is filtered by the at least one filtering component in the ventilation channel and discharged to the outdoor space, thereby the air pollution in the indoor space is rapidly exchanged and discharged to the outdoor space, and the air pollution in the indoor space is filtered by the at least one filtering component to allow the indoor air pollution data to be the safety detection value.

7. The system for detecting and cleaning indoor air pollution according to claim 1, wherein the filtering devices further comprise a heating, ventilation and air conditioning system.

8. The system for detecting and cleaning indoor air pollution according to claim 7, wherein the heating, ventilation and air conditioning system comprises a gate and a plurality of channels, the gate controls the channels to introduce the outdoor gas into the indoor space, wherein the channels comprise at least one blower and are in communication with the indoor space, the gate controls the outdoor gas to be guided into the channels by the at least one blower and filtered by the at least one filtering component; the channels have a return inlet adapted to introduce the indoor gas in the indoor space back into the channels to make the circulative filtration; the control central processor receives the outdoor air pollution data detected by the at least one outdoor gas detection device and the indoor air pollution data detected by the indoor gas detection devices to perform the intelligent computation; if the indoor air pollution data is greater than the outdoor air pollution data, the control central processor transmits the control command to the indoor gas detection device of the heating, ventilation and air conditioning system to control the gate to be opened and to enable the at least one blower, thereby the air pollution in the indoor space is exchanged and discharged to the outdoor space, and the circulative filtration and the rapid clean of the air pollution in the indoor space can be achieved by the at least one filtering component to allow the indoor air pollution data to be the safety detection value.

9. The system for detecting and cleaning indoor air pollution according to claim 1, wherein in the intelligent computation, the control central processor receives the outdoor air pollution data detected by the at least one outdoor gas detection device and the indoor air pollution data detected by the indoor gas detection devices by connecting to a cloud processing device through a wireless transmission so as to perform artificial intelligent (AI) computation and big data comparison to locate the air pollution location in the indoor space and transmit the control command intelligently and selectively.

10. The system for detecting and cleaning indoor air pollution according to claim 9, wherein in the intelligent computation, the control central processor receives a highest data among the indoor air pollution data through the cloud processing device to locate the air pollution location in the indoor space and transmit the control command intelligently and selectively.

11. The system for detecting and cleaning indoor air pollution according to claim 9, wherein in the intelligent computation, the control central processor receives the indoor air pollution data detected by at least three of the indoor gas detection devices through the cloud processing device to locate the air pollution location in the indoor space and transmit the control command intelligently and selectively.

12. The system for detecting and cleaning indoor air pollution according to claim 1, wherein the intelligent computation locate the air pollution location in the indoor space, the control command is intelligently and selectively transmitted to a filtering device at the air pollution location and rest of the filtering devices which are outside the air pollution location respectively, enabling the operations of the filtering devices at the air pollution location and outside the air pollution location to generate the air convection directed to the air pollution; the air convection accelerates the filtering of the air pollution at the air pollution location and the air pollution outside the air pollution location which is diffused, moved, and directed by the air convection, and the filtering components of the rest of the filtering devices outside the air pollution location are enabled intelligently and selectively, therefore the air pollution in the indoor space is filtered to allow the indoor air pollution data to be the safety detection value in which the air pollution data approaches to the non-detection state, and the gas in the indoor space is cleaned to the safe and breathable state.

13. The system for detecting and cleaning indoor air pollution according to claim 1, wherein the filtering component of one of the filtering devices is a physical-typed filtering device, and the filtering component filters the air pollution physically by a filter to block and absorb the air pollution.

14. The system for detecting and cleaning indoor air pollution according to claim 13, wherein the filter is a high-efficiency particulate air filter.

15. The system for detecting and cleaning indoor air pollution according to claim 1, wherein the filtering component of one of the filtering devices is a chemical-typed filtering device, and the filtering component filters the air pollution chemically by applying a degradation layer on the filtering component.

16. The system for detecting and cleaning indoor air pollution according to claim 15, wherein the degradation layer comprises at least one selected from the group consisting of an activated carbon, a cleansing factor layer having chlorine dioxide, an herbal protection coating layer including the extracts of Rhus chinensis Mill and the extracts of Ginkgo biloba, a layer of silver ions, a zeolite mesh, and any combination thereof.

17. The system for detecting and cleaning indoor air pollution according to claim 1, wherein the filtering component of one of the filtering devices is a chemical-typed filtering device, and the filtering component filters the air pollution chemically along with a light illumination.

18. The system for detecting and cleaning indoor air pollution according to claim 17, wherein the light illumination comprises at least one selected from the group consisting of a photocatalyst, a photocatalyst unit of an ultraviolet light, a photo plasma unit of a nanometer light tube, and any combination thereof.

19. The system for detecting and cleaning indoor air pollution according to claim 1, wherein the filtering component of one of the filtering devices is a chemical-typed filtering device, and the filtering component filters the air pollution chemically along with a degradation unit.

20. The system for detecting and cleaning indoor air pollution according to claim 19, wherein the degradation unit comprises at least one selected from the group consisting of a negative ion unit, a plasma ion unit, and any combination thereof.

21. The system for detecting and cleaning indoor air pollution according to claim 1, wherein each of the at least one outdoor gas detection device and the indoor gas detection devices is a gas detection device, the gas detection device comprises a control circuit board, a gas detection main body, a microprocessor, and a communication device; the gas detection main body, the microprocessor, and the communication device are integrally packaged and electrically connected to the control circuit board; the microprocessor controls the operation of the gas detection main body, the gas detection main body detects the air pollution and output a detection signal, and the microprocessor receives the detection signal to perform computation to generate the indoor air pollution data and the outdoor air pollution data and provides the indoor air pollution data and outdoor air pollution data to the communication device through a wireless transmission outwardly.

22. The system for detecting and cleaning indoor air pollution according to claim 21, wherein the gas detection main body comprises:
a base, having:
a first surface;
a second surface opposite to the first surface;
a laser installation region hollowed out from the first surface to the second surface;
a gas inlet groove recessed from the second surface and located adjacent to the laser installation region, wherein the gas inlet groove has a gas inlet through hole and two lateral walls; two light penetration windows penetrate on the two lateral walls of the gas inlet groove and are in communication with the laser installation region;
a gas-guiding component installation region recessed from the second surface and in communication with the gas inlet groove, wherein a ventilation hole penetrates a bottom surface of the gas-guiding component installation region; and
a gas outlet groove including a first region and a second region, wherein the first region is corresponding to the gas-guiding component installation region and is recessed from a portion of the first surface corresponding to a bottom surface of the gas-guiding component installation region; the second region is hollowed out from the first surface to the second surface in a region that is not corresponding to the gas-guiding component installation region; the gas outlet groove is in communication with the ventilation hole and has a gas outlet through hole;
a piezoelectric actuator received in the gas-guiding component installation region;
a driving circuit board covering and attached to the second surface of the base;
a laser component disposed on and electrically connected to the driving circuit board, wherein the laser component is received in the laser installation region, and a light path of a light beam emitted by the laser component passes through the light penetration windows and is orthogonal to the gas inlet groove;
a particulate sensor disposed on and electrically connected to the driving circuit board, wherein the particulate sensor is received in a position of the gas inlet groove where the path of the light beam emitted by the laser component is orthogonal to the gas inlet groove, so that the particulates in the air pollution passing through the gas inlet groove which is illuminated by the light beam of the laser component is detected by the particulate sensor;
a gas sensor disposed on and electrically connected to the driving circuit board, wherein the gas sensor is received in the gas outlet groove for detecting the air pollution introduced into the gas outlet groove; and
an outer cover covering the base and having a side plate, and the side plate has a gas inlet opening and a gas outlet opening, the gas inlet opening is corresponding to the gas inlet through hole of the base, and the gas outlet opening is corresponding to the gas outlet through hole of the base;
wherein when the outer cover is covered on the base and the driving circuit board is attached to the second surface of the base, a gas inlet path is defined by the gas inlet groove and a gas outlet path is defined by the gas outlet groove, thereby the piezoelectric actuator is driven to accelerate the introduction of the air pollution outside the gas inlet through hole into the gas inlet path defined by the gas inlet groove from the gas inlet opening; the air pollution passes through the particulate sensor to detect a particle concentration of the particulates contained in the air pollution; and the air pollution discharged into the gas outlet path defined by the gas outlet groove from the ventilation hole, detected by the gas sensor, and is discharged out of the gas detection main body from the gas outlet through hole and the gas outlet opening of the base.

23. The system for detecting and cleaning indoor air pollution according to claim 22, wherein the gas sensor comprises at least one selected from the group consisting of a volatile organic compound detector, a formaldehyde sensor, a bacterial sensor, a virus sensor, and any combination thereof; the volatile organic compound detector is capable of detecting information of carbon dioxide or total volatile organic compounds; the formaldehyde sensor is capable of detecting information of formaldehyde (HCHO) gas; the bacterial sensor is capable of detecting information of bacteria or fungi; the virus sensor is capable of detecting information of viruses.
